(19) United States
(12) Patent Application Publication
(10) Pub. No.: US 2013/0178753 A1
(43) Pub. Date: Jul. 11, 2013

(54) Heart Rate Monitor

(71) Applicant: Shing-Wai David WU, Kowloon (CN)

(72) Inventor: Shing-Wai David WU, Kowloon (CN)

(21) Appl. No.: 13/344,830

(22) Filed: Jan. 6, 2012

(51) Int. Cl. A61B 5/0255

(52) U.S. Cl. 600/508

(57) ABSTRACT

A heart rate monitoring system includes a wrist-borne heart rate monitor for detecting the heart rate of a wearer and having a mobile telephone network transmitter and/or WiFi transmitter for transmitting the heart rate data to the cloud. A display device includes a mobile telephone network receiver and/or WiFi receiver and receives the data and/or information processed from the data on the cloud and displays the same to the wearer.

### BACKGROUND OF THE INVENTION

The present invention relates to heart rate monitors. The invention more particularly, although not exclusively, relates to a wrist-mounted heart rate monitor that communicates indirectly with a mobile phone via the Internet.

It is known to transmit heart rate (pulse) information data directly from a wrist-borne heart rate monitoring device to a mobile phone (such as an iPhone™) via a local WiFi connection.

### SUMMARY

A heart rate monitor system transmits heart rate information from a heart rate monitor to a remote location (such as somewhere on the Internet) hereinafter referred to as “the cloud” for processing and/or storage and to monitor the information on a mobile phone or other display device in real time and or later on.

As used herein, the term “the cloud” is intended to mean a place or places accessed via the Internet and providing computation, software, data access and storage services that do not require end-user knowledge of the physical location and configuration of the system that delivers the services.

There is disclosed herein a heart rate monitoring system, comprising:

a heart rate monitor for detecting the heart rate of a wearer and having a mobile telephone network transmitter and/or WiFi transmitter for transmitting said heart rate data to the cloud; and

a display device including a mobile telephone network receiver and/or WiFi receiver receiving said data and/or information processed from said data on the cloud and displaying same.

Preferably, said heart rate monitor is incorporated into a wrist-borne device.

Preferably, said wrist-borne device is a wrist watch.

Preferably, said display device is incorporated into a mobile telephone.

### BRIEF DESCRIPTION OF THE DRAWINGS

A preferred form of the present invention will now be described by way of example with reference to the accompanying drawings, wherein:

FIG. 1 is a schematic illustration of a wrist-borne heart rate monitor upon the rest of a wearer;

FIG. 2 is a schematic illustration similar to FIG. 1, but with the wrist turned over;

FIG. 3 is a schematic illustration of the wrist-borne device showing internal features;

FIG. 4 is a schematic illustration of the wrist-borne device showing the removal/insertion of a micro-SIM;

FIG. 5 is a schematic illustration of the wrist-borne monitoring device with the micro-SIM installed; and

FIG. 6 is a schematic illustration of the monitoring device uploading data via a mobile network or WiFi connection to the cloud and also showing data being downloaded via the mobile network or WiFi connection to a telephone.

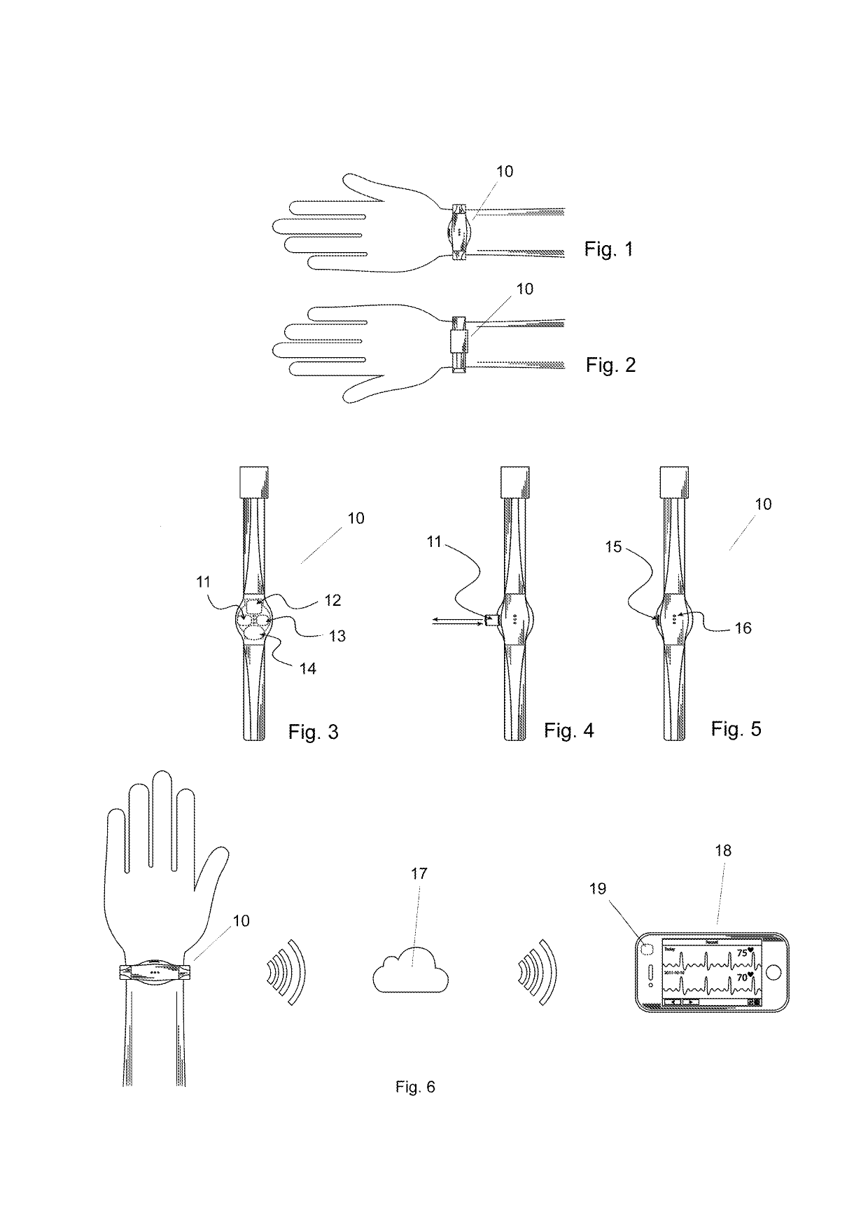

### DETAILED DESCRIPTION

In the accompanying drawings there is depicted schematically a heart rate monitor 10 incorporated into a wristband or wrist watch. A micro-SIM 11 can be inserted into a micro-SIM slot 15. LEDs 16 are provided upon the casing to indicate activity of the monitor 10.

Also located within the heart rate monitor 10 is a CPU 12, a pulse sensor 14 for detecting the pulse of the wearer, and a mobile telephone network transmitter and/or WiFi transmitter module 13. Also typically included but not depicted would be a button battery and a RAM module.

The transmitter 13 uploads/transmits the monitored heart rate data to the cloud 17 as indicated in FIG. 6 whereupon the data is stored and/or processed for download/transmission via the mobile network and/or WiFi network to a receiver module 19 of a mobile telephone 18 whereupon a cardiographic display and/or other information processed remotely and related to the pulse is displayed.

The system can be provided with a breach of tolerance feature to enact an alert notification based on a pre-defined healthy pulse rate range. The feature could be software-driven for regular updates and pre-timed updates.

The system is designed to invisibly monitor a wearer's condition. The monitoring person (typically the wearer or a nurse for example) sets the tolerance range based on either standard charts or a pre-defined tolerance based on knowledge and medical history of the individual wearer (the person being monitored).

The system constantly monitors the user's condition in the background and when the pulse rate falls outside the normal range (say for example: the normal adult geriatric heart rate is 70-95), a notification is automatically generated and sent via the Cloud to the display device.

This provides a comfort level to the monitoring person of the knowledge that the wearer is constantly monitored (24 hours a day). There is also a practical benefit of the monitoring person being disturbed/alerted only when there might be something wrong (breach of tolerance) of the wearer's monitored condition.

The heart rate monitor would typically be powered by a rechargeable battery and/or a button cell battery and an on/off switch. However a proximity sensor could be provided on the wrist device in lieu of an on/off switch so that when the device is attached to a wrist for example it would power-up automatically.

Typically, the whole data process is monitored and managed using an app (software program).

The wrist watch or wrist watch shaped device may or may not be waterproof.

It should be appreciated that modifications and alterations obvious to those skilled in the art are not to be considered as beyond the scope of the present invention. For example, the technology could equally be applicable to other monitorable bodily traits such as blood pressure.

1. A heart rate monitoring system, comprising:
a heart rate monitor for detecting the heart rate of a wearer and having a mobile telephone network transmitter and/or WiFi transmitter for transmitting said heart rate data to the cloud; and
a display device including a mobile telephone network receiver and/or WiFi receiver receiving said data and/or information processed from said data on the cloud and displaying same.

2. The system of claim 1, wherein said heart rate monitor is incorporated into a wrist-borne device.

3. The system of claim 2, wherein said wrist-borne device is a wrist watch.

4. The system of claim 1, wherein said display device is incorporated into a mobile telephone.
